(19) United States
(12) Patent Application Publication
(10) Pub. No.: US 2023/0420118 A1
(43) Pub. Date: Dec. 28, 2023

(54) TECHNOLOGIST ASSESSMENTS FOR PROFESSIONAL GROWTH AND OPERATIONAL IMPROVEMENT

(71) Applicant: KONINKLIJKE PHILIPS N.V., EINDHOVEN (NL)

(72) Inventors: Olga STAROBINETS, NEWTON, MA (US); Sandeep Madhukar DALAL, WINCHESTER, MA (US); Hareesh CHAMARTHI, CAMBRIDGE, MA (US)

(21) Appl. No.: 18/036,672

(22) PCT Filed: Nov. 9, 2021
(86) PCT No.: PCT/EP2021/081005

§ 371 Date: May 12, 2023

Related U.S. Application Data
(60) Provisional application No. 63/114,558, filed on Nov. 17, 2020.

(51) Int. Cl. G16H 40/20; G16H 40/63; G16H 80/00

(52) U.S. Cl. CPC G16H 40/20; G16H 80/00; G16H 40/63

(57) ABSTRACT

A method (100) of assessing performance of a local medical imaging device operator (LO) during imaging examinations performed using one or more medical imaging devices (2) includes: obtaining data related to the imaging examinations during performance of the medical imaging examinations; analyzing the obtained data to determine one or more performance metrics of the local operator; and during a current imaging examination, providing a remote assistance user interface (UI) (28) to at least one display device (24) operable by a remote expert (RE), the UI providing two-way communication between the local medical imaging device operator and the remote expert via which the remote expert can provide assistance to the local medical imaging device operator, the UI further displaying a visualization (34) of the determined one or more performance metrics of the local operator.

The following relates generally to the imaging arts, remote imaging assistance arts, remote imaging examination monitoring arts, technology assessment arts, technologist development arts, and related arts.

### BACKGROUND

Radiology operations command centers (ROCC) are a promising way for large imaging centers with pools of technologist (“tech”) talent to share tech expertise across an entire imaging network. By providing a communication channel (e.g. telephonic, videoconferencing, or so forth) and remote imaging device controller console sharing, an ROCC empowers the more experienced techs to provide guidance and oversight for junior techs when working with an imaging modality they may not be familiar or comfortable with. The quality of images acquired with ROCC assistance and the success achieved by use of an ROCC depends at least in part on how well the needs of the local techs are anticipated. However, assessing the knowledge and skills of individuals and evaluating their ability to perform the required tasks is difficult to do.

The following discloses certain improvements to overcome these problems and others.

### SUMMARY

In one aspect, a non-transitory computer readable medium stores instructions executable by at least one electronic processor to perform a method of assessing performance of a local medical imaging device operator during imaging examinations performed using one or more medical imaging devices. The method includes: obtaining data related to the imaging examinations during performance of the medical imaging examinations; analyzing the obtained data to determine one or more performance metrics of the local operator; and during a current imaging examination, providing a remote assistance user interface (UI) to at least one display device operable by a remote expert, the UI providing two-way communication between the local medical imaging device operator and the remote expert via which the remote expert can provide assistance to the local medical imaging device operator, the UI further displaying a visualization of the determined one or more performance metrics of the local operator.

In another aspect, an apparatus for use in conjunction with a medical imaging device includes at least one electronic processor programmed to: receive images of an imaging examination performed by a local operator and audio or textual conversations between the local operator and a remote medical expert during the imaging examination; obtain data related to the imaging examinations during performance of the medical imaging examinations from the recorded images and recorded conversations; analyze the obtained data to determine one or more performance metrics of the local operator; and during a current imaging examination, provide a remote assistance user UI to at least one display device operable by a remote expert, the UI providing two-way communication between the local medical imaging device operator and the remote expert via which the remote expert can provide assistance to the local medical imaging device operator, the UI further displaying a visualization of the determined one or more performance metrics of the local operator.

In another aspect, a method of assessing performance of a local medical imaging device operator during imaging examinations performed using one or more medical imaging devices includes: screen-scraping data displayed on a display device of a medical imaging device controller of the medical imaging device; analyzing the screen-scraped data to determine one or more performance metrics of the local operator; and during a current imaging examination, providing a remote assistance UI to at least one display device operable by a remote expert, the UI providing two-way communication between the local medical imaging device operator and the remote expert via which the remote expert can provide assistance to the local medical imaging device operator, the UI further displaying a visualization of the determined one or more performance metrics of the local operator.

One advantage resides in automatically providing a remote expert or radiologist assisting a technician in conducting a medical imaging examination with information about the local technician.

Another advantage resides in assessing a technician's knowledge, skills, and limitations for medical examinations, and accordingly matching a remote expert with the technician.

Another advantage resides in tracking performance of a technician in performing imaging examinations, and tracking progress of the technician towards accreditation requirements.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 shows an example flow chart of operations suitably performed by the apparatus of FIG. 1.

FIGS. 3, 4, 5A, 5B, and 6 show examples of outputs generated by the apparatus of FIG. 1.

### DETAILED DESCRIPTION

The following relates to Radiology Operations Command Center (ROCC) systems and methods that provide remote expert or “supertech” assistance to a local technician performing an imaging examination. An ROCC collects information on the imaging examination being performed by the local technician, which is supplied to the supertech to enable the supertech to provide effective assistance. The provided information typically includes, for example, a copy of the imaging device controller display. It is recognized herein that this collected information can be leveraged for other purposes.

In some embodiments disclosed herein, a database is included that collects information on individual imaging examinations, including for example identification of the imaging technician (also referred to herein as the local technician), the imaging procedure, imaging modality and scanner used, date and duration of the imaging examination, the assigned Current Procedural Terminology (CPT) code, identification of whether expert assistance was utilized, an image quality assessment, patient feedback, various special notes (e.g., wheelchair-bound patient, infant or elderly patient, chronic conditions of the patient, etc.) and/or so forth.

Most of this information can be obtained by screen-scraping the image of the controller display acquired by the DVI splitter, software-based screen mirroring, or other screen-sharing mechanism. Image quality assessment is further useful information, which optionally may be obtained from a radiologist self-assessment and/or automated image quality classification. Patient feedback still further useful information that is optionally obtained from post-examination surveys (for example, conducted via the Medumo cellphone app, available from Medumo, Cambridge, Massachusetts, USA).

With this information collected, various analyses of technician can be performed. For example, technologist expertise in various imaging procedures/modalities/vendors can be assessed statistically based on (for example) number of such imaging procedures the technologist has performed, exam durations (long durations may indicate the technician had difficulty with the exams), and the extent to which the technician relied upon the ROCC (more reliance may indicate low technician comfort with that type of imaging procedure). Technician performance can be analyzed longitudinally to see how rapidly the technician is improving.

Furthermore, the collected information can be used for diverse tasks such as providing a metric for determining technician advancement, identifying examinations needed to for accreditation, identifying optimal training areas for a given technician, and so forth.

A further use of the information is to generate technician summaries or profiles. These succinctly summarize the technician's experience in various types of imaging tasks as well as areas where the technician may be likely to need assistance. In the ROCC context, when an expert is called by a particular local technician, the summary or profile of that local technician may be displayed on the expert workstation so that the expert is quickly brought up to speed on the abilities and possible deficiencies of the local technician.

As yet another application, in some embodiments each imaging examination is recorded. This exam record includes a video of the scraped screen and a transcript or recording of any audio, videoconference, and/or chat box conversation that occurred between the local technician and the expert during the exam. These exam records can serve as ready-made training materials, e.g. if a particular local technician is determined to require training in some imaging procedure “X”, then the database of exam records can be searched for the best examples of imaging procedure “X” as measured by quantitative metrics such as image quality, patient feedback, exam duration, and/or so forth.

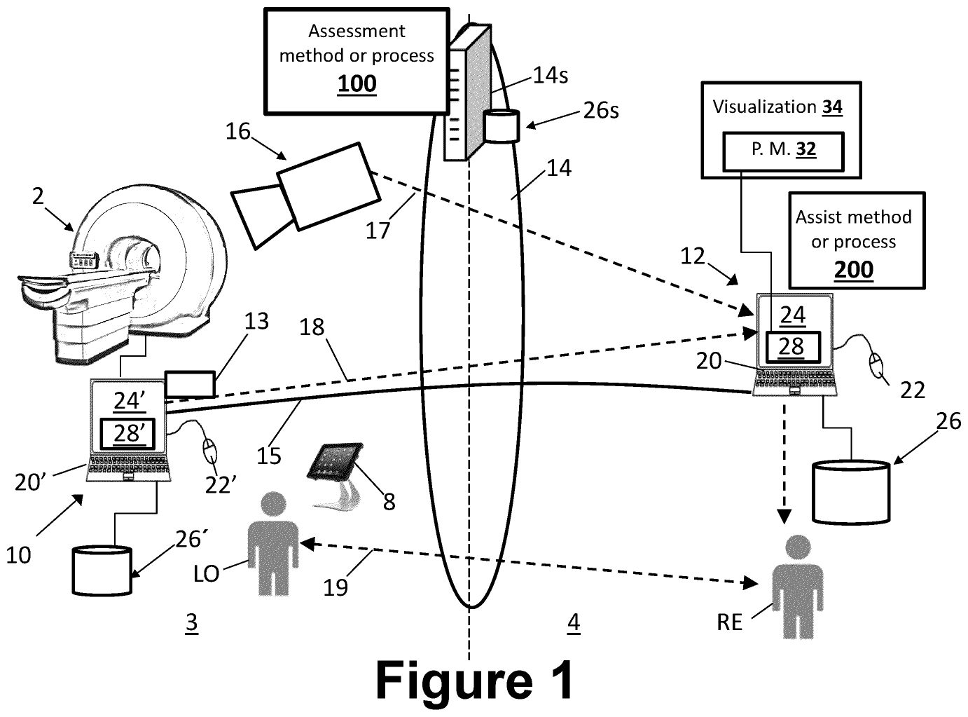

With reference to FIG. 1, an apparatus for providing assistance from a remote medical imaging expert RE (or supertech) to a local technician operator LO is shown. Such a system is also referred to herein as a radiology operations command center (ROCC). As shown in FIG. 1, the local operator LO, who operates a medical imaging device (also referred to as an image acquisition device, imaging device, and so forth) 2, is located in a medical imaging device bay 3, and the remote expert RE is disposed in a remote service location or center 4. It should be noted that the remote expert RE may not necessarily directly operate the medical imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the medical imaging device bay 3 (this may, for example, in the case of a remote expert RE who is a radiologist tasked with peri-examination image review), or the remote service center 4 and the medical imaging device bay 3 may be in different buildings, and indeed may be located in different cities, different countries, and/or different continents. In general, the remote location 4 is remote from the imaging device bay 3 in the sense that the remote expert RE cannot directly visually observe the imaging device 2 in the imaging device bay 3 (hence optionally providing a video feed as described further herein).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities. For example, if a hospital performs many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. This is merely an example. Moreover, the remote service center 4 may provide service to multiple hospitals. The local operator LO controls the medical imaging device 2 via an imaging device controller 10. The remote expert RE is stationed at a remote workstation 12 (or, more generally, an electronic controller 12).

As used herein, the term “medical imaging device bay” (and variants thereof) refer to a room containing the medical imaging device 2 and also any adjacent control room containing the medical imaging device controller 10 for controlling the medical imaging device. For example, in reference to an MRI device, the medical imaging device bay 3 can include the radiofrequency (RF) shielded room containing the MRI device 2, as well as an adjacent control room housing the medical imaging device controller 10, as understood in the art of MRI devices and procedures. On the other hand, for other imaging modalities such as CT, the imaging device controller 10 may be located in the same room as the imaging device 2, so that there is no adjacent control room and the medical bay 3 is only the room containing the medical imaging device 2. The imaging device controller 10 includes an electronic processor 20′, at least one user input device such as a mouse 22′, a keyboard, and/or so forth, and a display device 24′. The imaging device controller 10 presents a device controller graphical user interface (GUI) 28′ on the display 24′ of the imaging device controller 10, via which the local operator LO accesses device controller GUI screens for entering the imaging examination information such as the name of the local operator LO, the name of the patient and other relevant patient information (e.g. gender, age, etc.) and for controlling the (typically robotic) patient support to load the patient into the bore or imaging examination region of the imaging device 2, selecting and configuring the imaging sequence(s) to be performed, acquiring preview scans to verify positioning of the patient, executing the selected and configured imaging sequences to acquire clinical images, display the acquired clinical images for review, and ultimately store the final clinical images to a Picture Archiving and Communication System (PACS) or other imaging examinations database. In addition, while FIG. 1 shows a single medical imaging device bay 3, it will be appreciated that the remote service center 4 (and more particularly the remote workstation 12) is in communication with multiple medical bays via a communication link 14, which typically comprises the Internet augmented by local area networks at the remote operator RE and local operator LO ends for electronic data communications.

As diagrammatically shown in FIG. 1, in some embodiments, a camera 16 (e.g., a video camera) is arranged to acquire a video stream 17 of a portion of the medical imaging device bay 3 that includes at least the area of the imaging device 2 where the local operator LO interacts with the patient, and optionally may further include the imaging device controller 10. The video stream 17 is sent to the remote workstation 12 via the communication link 14, e.g. as a streaming video feed received via a secure Internet link. In some examples, as shown in FIG. 1, the camera 16 can be affixed to a wall of ceiling of the medical facility with a field of view to include the area of the imaging device 2 where the local operator LO interacts with the patient, and optionally may further include the imaging device controller 10. In other examples, the camera 16 can be disposed within an imaging bore (not shown) of the imaging device 2.

In other embodiments, the live video feed 17 of the display 24′ of the imaging device controller 10 is, in the illustrative embodiment, provided by a video cable splitter 15 (e.g., a DVI splitter, a HDMI splitter, and so forth). In other embodiments, the live video feed 17 may be provided by a video cable connecting an auxiliary video output (e.g. aux vid out) port of the imaging device controller 10 to the remote workstation 12 of the operated by the remote expert RE. Alternatively, a screen mirroring data stream 18 is generated by screen sharing software 13 running on the imaging device controller 10 which captures a real-time copy of the display 24′ of the imaging device controller 10, and this copy is sent from the imaging device controller 10 to the remote workstation 12. Other approaches besides the illustrative video cable splitter 15 or screen sharing software 13 are contemplated for capturing a real-time copy of the display 24′ of the imaging device controller 10 which is then sent to the workstation 12 of the remote expert RE. While in an ROCC this real-time copy of the display 24′ of the imaging device controller 10 is used to provide status information to the remote expert RE for use in assisting the local operator LO, in embodiments disclosed herein the real-time copy of the display 24′ of the imaging device controller 10 is also leveraged (optionally along with other available information) to determine one or more performance metrics of the local operator LO.

The communication link 14 also provides a natural language communication pathway 19 for verbal and/or textual communication between the local operator LO and the remote expert RE, in order to enable the latter to assist the former in performing the imaging examination. For example, the natural language communication link 19 may be a Voice-Over-Internet-Protocol (VOIP) telephonic connection, a videoconferencing service, an online video chat link, a computerized instant messaging service, or so forth. Alternatively, the natural language communication pathway 19 may be provided by a dedicated communication link that is separate from the communication link 14 providing the data communications 17, 18, e.g. the natural language communication pathway 19 may be provided via a landline telephone. In another example, the natural language communication pathway 19 may be provided via an ROCC device 8, such as a mobile device (e.g., a tablet computer or a smartphone). For example, an “app” can run on the ROCC device 8 (operable by the local operator LO) and the remote workstation 12 (operable by the remote expert RE) to allow communication (e.g., audio chats, video chats, and so forth) between the local operator and the remote expert.

FIG. 1 also shows, in the remote service center 4 including the remote workstation 12, such as an electronic processing device, a workstation computer, or more generally a computer, which is operatively connected to receive and present the video 17 of the medical imaging device bay 3 from the camera 16 and to present the screen mirroring data stream 18 as a mirrored screen. Additionally or alternatively, the remote workstation 12 can be embodied as a server computer or a plurality of server computers, e.g. interconnected to form a server cluster, cloud computing resource, or so forth. The workstation 12 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, cathode ray tube display, and/or so forth). In some embodiments, the display device 24 can be a separate component from the workstation 12. The display device 24 may also comprise two or more display devices, e.g. one display presenting the video 17 and the other display presenting the shared screen (i.e. display 24′) of the imaging device controller 10 generated from the screen mirroring data stream 18. Alternatively, the video and the shared screen may be presented on a single display in respective windows. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24.

The medical imaging device controller 10 in the medical imaging device bay 3 also includes similar components as the remote workstation 12 disposed in the remote service center 4. Except as otherwise indicated herein, features of the medical imaging device controller 10 disposed in the medical imaging device bay 3 similar to those of the remote workstation 12 disposed in the remote service center 4 have a common reference number followed by a “prime” symbol (e.g., processor 20′, display 24′, GUI 28′) as already described. In particular, the medical imaging device controller 10 is configured to display the imaging device controller GUI 28′ on a display device or controller display 24′ that presents information pertaining to the control of the medical imaging device 2 as already described, such as imaging acquisition monitoring information, presentation of acquired medical images, and so forth. It will be appreciated that the real-time copy of the display 24′ of the controller 10 provided by the video cable splitter 15 or the screen mirroring data stream 18 carries the content presented on the display device 24′ of the medical imaging device controller 10. The communication link 14 allows for screen sharing from the display device 24′ in the medical imaging device bay 3 to the display device 24 in the remote service center 4. The GUI 28′ includes one or more dialog screens, including, for example, an examination/scan selection dialog screen, a scan settings dialog screen, an acquisition monitoring dialog screen, among others. The GUI 28′ can be included in the video feed 17 or provided by the video cable splitter 15 or by the mirroring data stream 17′ and displayed on the remote workstation display 24 at the remote location 4.

FIG. 1 shows an illustrative local operator LO, and an illustrative remote expert RE (i.e. expert, e.g. supertech). However, the ROCC optionally provides a staff of supertechs who are available to assist local operators LO at different hospitals, radiology labs, or the like. The ROCC may be housed in a single physical location or may be geographically distributed. For example, in one contemplated implementation, the remote operators RO are recruited from across the United States and/or internationally in order to provide a staff of supertechs with a wide range of expertise in various imaging modalities and in various imaging procedures targeting various imaged anatomies. In view of this multiplicity of local operators LO and multiplicity of remote operators RO, the disclosed communication link 14 includes a server computer 14s (or a cluster of servers, cloud computing resource comprising servers, or so forth) which is programmed to establish connections between selected local operator LO/remote expert RE pairs. For example, if the server computer 14s is Internet-based, then connecting a specific selected local operator LO/remote expert RE pair can be done using Internet Protocol (IP) addresses of the various components 16, 10, 12, the telephonic or video terminals of the natural language communication pathway 19, et cetera. The server computer 14s is operatively connected with a one or more non-transitory storage media 26s. The non-transitory storage media 26s may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14s, various combinations thereof, or so forth. It is to be understood that any reference to a non-transitory medium or media 26s herein is to be broadly construed as encompassing a single medium or multiple media of the same or different types. Likewise, the server computer 14s may be embodied as a single electronic processor or as two or more electronic processors. The non-transitory storage media 26s stores instructions executable by the server computer 14s. In addition, the non-transitory computer readable medium 26s (or another database) stores data related to a set of remote experts RE and/or a set of local operators LO. The remote expert data can include, for example, skill set data, work experience data, data related to ability to work on multi-vendor modalities, data related to experience with the local operator LO and so forth.

Furthermore, as disclosed herein the server 14s performs a method or process 100 for assessing performance of a local medical imaging device operator LO during imaging examinations performed using one or more of the medical imaging devices 2. The assessment method 100 advantageously leverages information sources provided by the ROCC, such as the content of the display 24′ of the imaging device controller display

The server computer 14s can also store data related to relevant information acquired during each imaging examination performed by a local operator LO. The stored data can include, for example, a vendor and modality of the medical imaging device 2 used in the imaging examination, an identification of the local medical imaging device operator LO, a modality of the medical imaging device an identification of the medical imaging device, an assigned current procedural terminology code, an identification of whether assistance from a remote medical expert was used, communication notes between the local operator and the remote medical expert, patient data (e.g., frail individuals, pediatric exams, disabled individuals, and so forth), a duration of the imaging examination, sequences repeated during the imaging examination, quality of images acquired during the imaging examination, notes regarding adversary events (e.g., device malfunction), and so forth.

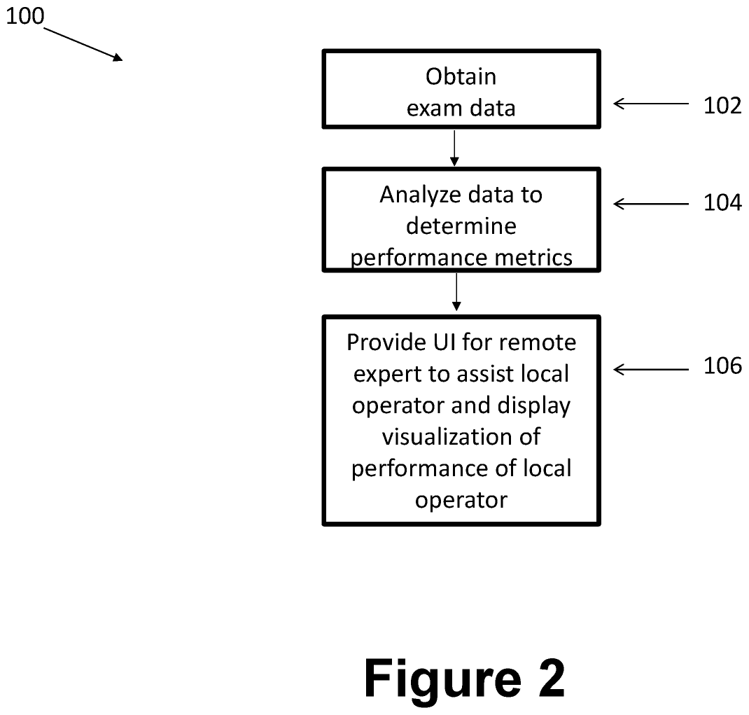

With reference to FIG. 2, and with continuing reference to FIG. 1, an illustrative embodiment of the technician assessment method 100 is diagrammatically shown as a flowchart. At an operation 102, data related to the imaging examinations is obtained during performance of the medical imaging examinations. In one example, the obtaining operation 102 includes screen-scraping data displayed on the display device 24′ of the medical imaging device controller 10 of the medical imaging device 2. This screen-scraping leverages the availability of the real-time copy of the display 24′ of the imaging device controller 10 provided by the video cable splitter 15 or the screen sharing software 13. The screen scraping can use any suitable approach for extracting relevant information from the real-time copy of the display 24′ of the imaging device controller 10. For example, video frames can be analyzed by optical character recognition (OCR) to extract text. As the imaging device controller GUI 28′ typically uses standardized dialog screens, the screen scraping can leverage a priori knowledge about the layouts of these dialog screens to enable more precise information extraction. For example, if a dialog screen has one input area for entry of the local operator LO identification and another input area for entry of the patient identification, then this a priori knowledge of the layout can be used to distinguish the local operator and patient names. Similarly, specific dialog screens may be brought up for specific imaging sequences, and recognition of these specific dialog screens in the real-time copy of the display 24′ of the imaging device controller 10 enables extraction of the selected imaging sequence, and a priori knowledge of the layouts of these dialog screens can be used to correlate numeric or other inputs to specific scan parameters. These are merely some non-limiting illustrative examples of information extraction approaches suitably used in the screen-scraping. The data displayed on the display device 24′ of a medical imaging device controller 10 typically includes information useful to the technician assessment method 100 such as an identification of the local medical imaging device operator LO, a modality of the medical imaging device 2, an identification of the medical imaging device, an assigned current procedural terminology code, an identification of whether assistance from a remote medical expert was used, and patient data.

In another example, the obtaining operation 102 includes recording images of an imaging examination performed by the local operator LO with the camera 16, and recording audio or textual conversations between the local operator and the remote medical expert during the imaging examination via the natural language communication pathway 19. The data can then be obtained from the recorded images and recorded conversations. The obtained data can also be stored in the server computer 14s.

The foregoing examination data gathering operation 102 is suitably performed each time the local operator LO performs an imaging examination with the ROCC in operation. In some embodiments, the ROCC operation is modified to generate the real-time copy of the display 24′ of the controller 10 provided by the video cable splitter 15 or the screen mirroring data stream 18 throughout each examination, even if the local operator LO does not utilize assistance of a remote expert via the ROCC. In this way, the existing hardware of the ROCC (e.g., the video cable splitter 15 or the screen mirroring software 13) is leveraged to ensure that examination data collection occurs for all examinations, regardless of whether and for how long the ROCC is utilized. The examination data collected over days, weeks, months, or longer are suitably collected to provide a sizable database of examination data for the local operator LO that may include many dozens, hundreds, or more imaging examinations performed by the local operator LO.

At an operation 104, this sizable database of examination data for the local operator LO collected using the ROCC infrastructure is analyzed to determine one or more performance metrics 32 of the local operator LO. In one example embodiment, the obtained data can be statistically assessed based on one or more of, for example, for the imaging examinations the local operator has performed, durations of imaging procedures the local operator has performed, an extent which the local operator accessed the GUI 28 for assistance to perform the imaging procedures, and so forth. From this analysis, the one or more performance metrics 32 can be determined, and can include, for example, a performance metric for determining advancement of the local operator LO, a performance metric for determining optimal imaging tasks to assign to the local operator, a performance metric for identifying imaging examinations needed for accreditation for the local operator, among others.

At an operation 106, during a current imaging examination, the GUI 28 is provided as a remote assistance UI on the display device 24 operable by a remote expert RE. The UI 28 provides two-way communication between the local operator LO and the remote expert RE via which the remote expert can provide assistance to the local medical imaging device operator. The UI 28 further displays a visualization 34 of the determined one or more performance metrics 32 of the local operator LO. The visualization 34 is representative of a profile summarizing performance of the local operator LO in performing the imaging examinations.

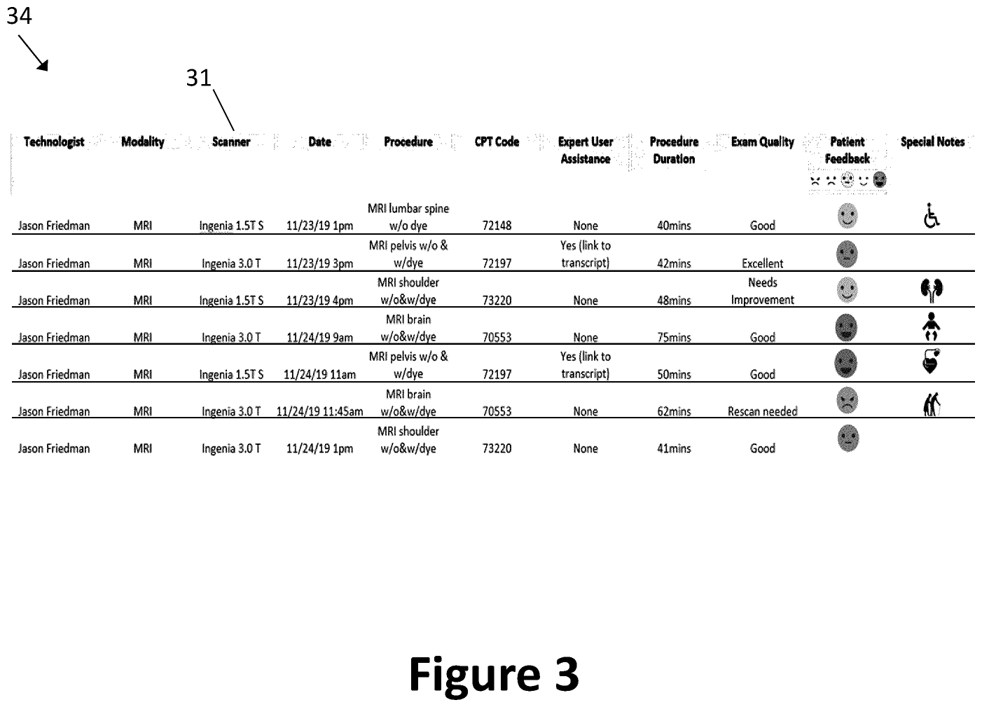

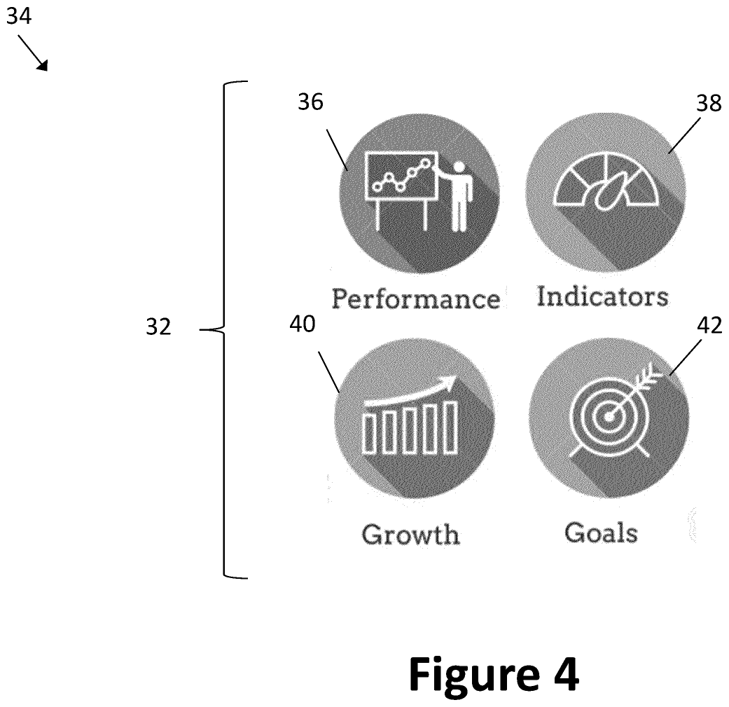

FIGS. 3, 4, 5, 5A, 5B, and 6 show examples the visualization 34. FIG. 3 shows an example of the data stored in the training database 31. FIG. 4 shows the visualization 34 including the performance metrics 32. A first performance metric 32 can include a “performance” icon 36 that includes a single local operator's LO performance for specific imaging examinations, or the performance of multiple local operators for a specific type of examination. This performance metric can include an option of identifying imaging examinations with poor patient feedback, extended procedure durations, and so forth for further analysis. A second performance metric 32 can include an “indicators” icon 38 that includes key performance indicators (KPIs) indicative of performance of a specific local operator LO compared to other, similar local operators. This performance metric 32 can be used to identify local operators LO for advancement (i.e., from junior tech to senior tech).

A third performance metric 32 can include a “growth” icon 40 including analytics of local operator LO performance over a selected time duration (e.g., six months, twelve months, etc.) to show improvements and identify areas that require further attention for particular local operator. A fourth performance metric 32 can include a “goals” icon 42 for setting goals for local operators LO based on their performance (i.e. improving proficiency in breast imaging, start scanning pediatric patients, work toward shortening procedure durations for Musculo-Skeletal (MSK) scans). Training opportunities can be identified based on the performance and goals of individual local operators LO.

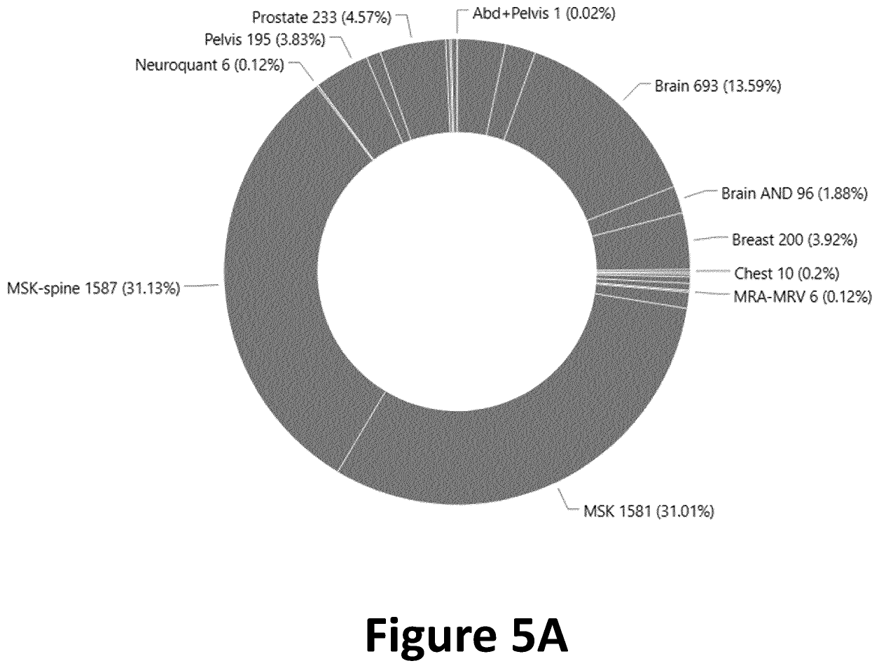

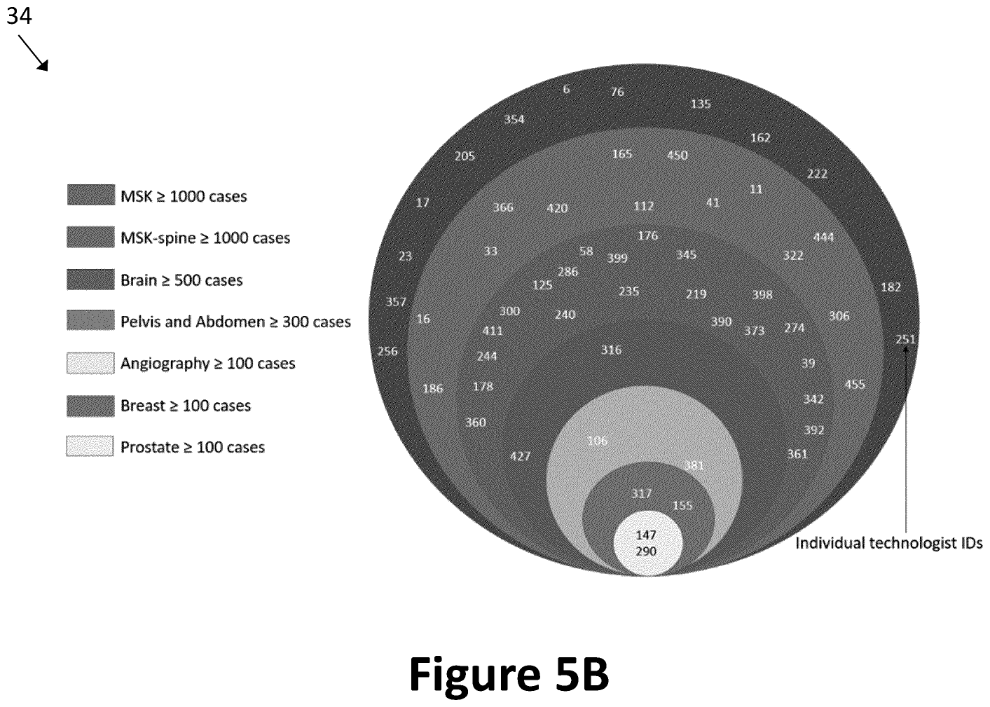

FIGS. 5A and 5B shows two more examples of the visualization 34. In a first example (FIG. 5A), a circle chart is shown with statistics on various types of imaging examinations performed by local operators LO. In a second example (FIG. 5B), a Venn diagram is shown based on types of examinations (in which the “largest circle” corresponds to the top rectangle in the key, the second-largest circle corresponds to the “second” rectangle in the key, and so forth).

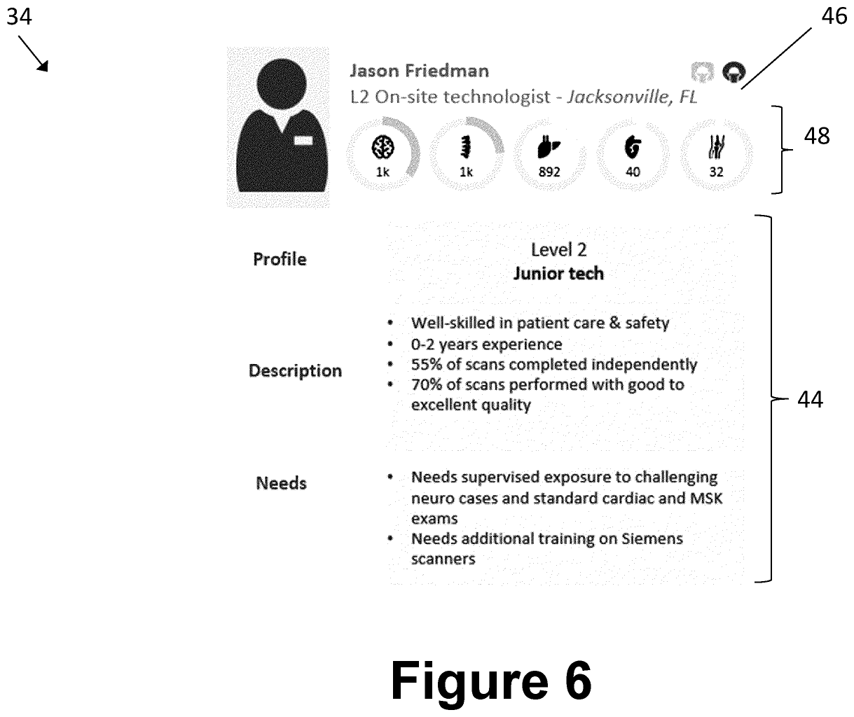

FIG. 6 shows an example of the visualization 34 of an individual local operator LO. A drop-down menu 46 listing a set of experience metrics 48 of the local operator LO can be shown. As shown in FIG. 6, the set of experience metrics 48 can include a brain imaging experience metric, a spine imaging experience metric, a liver imaging experience metric, a heart imaging experience metric, and a knee imaging experience metric. For instance, if a local operator LO has been noted to struggle in acquiring spine imaging (e.g., MRI) examinations on a medical imaging device 2 manufactured by Siemens scanner, a session demonstrating a past successful acquisition of the exact exam in question can be readily arranged.

FIG. 6 also shows a menu 44 showing a profile of the local operator LO, a description of the local operator's qualifications, and areas the local operator needs to improve on during imaging examinations. As an example, clinical experience requirements for MRI can include conducting 53 procedures in 7 different categories (e.g., head and neck, spine, thorax, abdomen and pelvis, msk, special imaging procedures, quality control). There are rules about the number of repetitions for different procedures (i.e., at least 125 repetitions across all procedures) and further nuances for different credential options. The apparatus 10 help techs and their managers to keep track of where they are in the accreditation process and make suggestions regarding upcoming scans matching technologist needs to complete the requirements within the predefined time window.

Referring back to FIG. 1, the communication link 14 connects the local operator LO/remote expert RE. The remote workstation 12 of the selected remote expert RE, and/or the medical imaging device controller 10 being run by the local operator LO, is configured to perform a method or process 200 for providing assistance from the remote expert RE to the local operator LO. For brevity, the method 200 will be described as being performed by the remote workstation 12. The non-transitory storage medium 26 stores instructions which are readable and executable by the at least one electronic processor 20 (of the workstation 12, as shown, and/or the electronic processor or processors of a server or servers on a local area network or the Internet) to perform disclosed operations including performing the method or process 200.

A suitable implementation of the assistance method or process 200 is as follows. The method 200 is performed over the course of (at least a portion of) a medical imaging examination performed using the medical imaging device 2, and the local expert RE is one selected via the matching method 100. As used herein, the term “duration of a medical imaging examination” (or variants thereof) refers to a time period of a medical imaging examination that includes (i) an actual image acquisition time, (ii) imaging follow-on processing time, and (iii) up to a time of patient release. To perform the method 200, the workstation 12 in the remote location 4 is programmed to receive at least one of: (i) the video 17 from the video camera 16 of the medical imaging device 2 located in the medical imaging device bay 3; and/or (ii) the screen sharing 18 from the screen sharing software 13; and/or (iii) the video 17 tapped by the video cable splitter 15. The video feed 17 and/or the screen sharing 18 can be displayed at the remote workstation display 24, typically in separate windows of the GUI 28. The video feed 17 and/or the screen sharing 18 can be screen-scraped to determine information related to the medical imaging examination (e.g., modality, vendor, anatomy to be imaged, cause of issue to be resolved, and so forth). In particular, the GUI 28 presented on the display 24 of the remote workstation 12 preferably includes a window presenting the video 17, and a window presenting the mirrored screen of the medical imaging device controller 10 constructed from the screen mirroring data stream 18, and status information on the medical imaging examination that is maintained at least in part using the screen-scraped information. This allows the remote operator RE to be aware of the content of the display of the medical imaging device controller 10 (via the shared screen) and also to be aware of the physical situation, e.g. position of the patient in the medical imaging device 2 (via the video 17), and to additionally be aware of the status of the imaging examination as summarized by the status information. During an imaging procedure, the natural language communication pathway 19 is suitably used to allow the local operator LO and the remote operator RE to discuss the procedure and in particular to allow the remote operator to provide advice to the local operator.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method of assessing performance of a local medical imaging device operator (LO) during imaging examinations performed using one or more medical imaging devices, the method comprising:
obtaining data related to the imaging examinations during performance of the medical imaging examinations;
analyzing the obtained data to determine one or more performance metrics of the local operator; and
during a current imaging examination, providing a remote assistance user interface (UI) to at least one display device operable by a remote expert (RE), the UI providing two-way communication between the local medical imaging device operator and the remote expert via which the remote expert can provide assistance to the local medical imaging device operator, the UI further displaying a visualization of the determined one or more performance metrics of the local operator.

2. The non-transitory computer readable medium of claim 1, wherein obtaining data related to the imaging examination includes:
screen-scraping data displayed on a display device of a medical imaging device controller of the medical imaging device.

3. The non-transitory computer readable medium of claim 2, wherein the data displayed on the display device of a medical imaging device controller includes an identification of the local medical imaging device operator (LO), a modality of the medical imaging device, an identification of the medical imaging device, an assigned current procedural terminology code, an identification of whether assistance from a remote medical expert was used, and patient data.

4. The non-transitory computer readable medium of claim 3, wherein analyzing the obtained data to determine one or more performance metrics of the local operator (LO) includes:
statistically assessing the obtained data for the imaging examinations the local operator has performed.

5. The non-transitory computer readable medium of claim 3, wherein analyzing the obtained data to determine one or more performance metrics of the local operator (LO) includes:
statistically assessing the obtained data based on durations of imaging procedures the local operator has performed.

6. The non-transitory computer readable medium of claim 3, wherein analyzing the obtained data to determine one or more performance metrics of the local operator (LO) includes:
statistically assessing the obtained data based on an extent which the local operator accessed the remote assistance UI for assistance to perform the imaging procedures.

7. The non-transitory computer readable medium of claim 1, wherein analyzing the obtained data to determine one or more performance metrics of the local operator (LO) includes:
from the analyzing, determining a performance metric for determining advancement of the local operator.

8. The non-transitory computer readable medium of claim 1, wherein analyzing the obtained data to determine one or more performance metrics of the local operator (LO) includes:
from the analyzing, determining a performance metric for determining optimal imaging tasks to assign to the local operator.

9. The non-transitory computer readable medium of claim 1, wherein analyzing the obtained data to determine one or more performance metrics of the local operator (LO) includes:
from the analyzing, determining a performance metric for identifying imaging examinations needed for accreditation for the local operator.

10. The non-transitory computer readable medium of claim 1, wherein the visualization is representative of a profile summarizing performance of the local operator (LO) in performing the imaging examinations.

11. The non-transitory computer readable medium of claim 1, wherein the method further includes:
recording images of an imaging examination performed by the local operator (LO);
recording audio or textual conversations between the local operator and a remote medical expert during the imaging examination; and
obtaining the data from the recorded images and recorded conversations.

12. The non-transitory computer readable medium of claim 11, wherein the method further includes:
storing the obtained data in a training database.

13. An apparatus for use in conjunction with a medical imaging device, the apparatus including at least one electronic processor programmed to:
receive images of an imaging examination performed by a local operator (LO) and audio or textual conversations between the local operator and a remote medical expert during the imaging examination;
obtain data related to the imaging examinations during performance of the medical imaging examinations from the recorded images and recorded conversations;
analyze the obtained data to determine one or more performance metrics of the local operator; and
during a current imaging examination, provide a remote assistance user interface (UI) to at least one display device operable by a remote expert (RE), the UI providing two-way communication between the local medical imaging device operator and the remote expert via which the remote expert can provide assistance to the local medical imaging device operator, the UI further displaying a visualization of the determined one or more performance metrics of the local operator.

14. The apparatus of claim 13, wherein the at least one electronic processor is programmed to:
screen-scrape data displayed on a display device of a medical imaging device controller of the medical imaging device.

15. The apparatus of claim 14, wherein the data displayed on the display device of a medical imaging device controller includes an identification of the local medical imaging device operator (LO), a modality of the medical imaging device, an identification of the medical imaging device, an assigned current procedural terminology code, an identification of whether assistance from a remote medical expert was used, and patient data.

16. The apparatus of claim 15, wherein the at least one electronic processor is programmed to statistically assess the obtained data for the imaging examinations the local operator has performed based on one or more of durations of imaging procedures the local operator has performed and an extent which the local operator accessed the remote assistance UI for assistance to perform the imaging procedures.

17. The apparatus of claim 13, wherein the at least one electronic processor is programmed to determining one or more performance metrics including a performance metric for determining advancement of the local operator, a performance metric for determining optimal imaging tasks to assign to the local operator, and a performance metric for identifying imaging examinations needed for accreditation for the local operator.

18. The apparatus of claim 13, wherein the visualization is representative of a profile summarizing performance of the local operator (LO) in performing the imaging examinations.

19. The apparatus of claim 13, further including:
a video cable splitter operatively connected with an imaging device controller of the medical imaging device via which the at least one electronic processor receives the data displayed on the imaging device controller.

20. A method of assessing performance of a local medical imaging device operator during imaging examinations performed using one or more medical imaging devices, the method comprising:
screen-scraping data displayed on a display device of a medical imaging device controller of the medical imaging device;
analyzing the screen-scraped data to determine one or more performance metrics of the local operator; and
during a current imaging examination, providing a remote assistance user interface (UI) to at least one display device operable by a remote expert (RE), the UI providing two-way communication between the local medical imaging device operator and the remote expert via which the remote expert can provide assistance to the local medical imaging device operator, the UI further displaying a visualization of the determined one or more performance metrics of the local operator.
